(19) United States
(12) Patent Application Publication
(10) Pub. No.: US 2012/0064145 A1
(43) Pub. Date: Mar. 15, 2012

(54) WOUND DRESSING

(75) Inventors: YU-SHIN LIN, Taichung City (TW); CHIA-LIN YEH, Taichung City (TW)

(73) Assignee: CHINA MEDICAL UNIVERSITY, Taichung (TW)

(21) Appl. No.: 13/015,301

(22) Filed: Jan. 27, 2011

(30) Foreign Application Priority Data
Sep. 9, 2010 (TW) 099130562

(51) Int. Cl. A61L 15/44; A61K 47/42; A61K 31/7036; A61K 35/12; A61K 31/65; A61K 31/43; A61K 31/18; A61K 36/00; A61K 31/165

(52) U.S. Cl. 424/445; 424/725; 514/774; 514/773; 514/777; 514/779; 514/40; 514/628; 514/152; 514/192; 514/603; 514/196; 424/93.7

(57) ABSTRACT

The present invention discloses a wound dressing which may be a double-layer wound dressing, comprising a polymer material layer in combination with anti-infective antibiotics or anti-infective Chinese herb medicine at the upper layer of the wound dressing, and a porous carbon material layer capable of loading with skin-associated epithelial cells at the lower layer of the wound dressing. The wound dressing with the antibiotics can block effectively the invasion of bacteria in the external environment, thereby preventing the wound from secondary infection. While covering the wound site, the porous carbon material layer with the epithelial cells can thus provide the healthy epithelial cells and attract the surrounding healthy cells to aggregate for secreting collagen so as to promote the wound healing process. According to the present invention, there is an obviously enhancing effect on wound tissue regeneration, restoration and healing, and there is a grant development potential in clinical applications.

### BACKGROUND OF THE INVENTION

1. Field of the Invention

The present invention relates to a wound dressing, and more particularly to a wound dressing capable of protecting from infections and promoting and accelerating wound healing concurrently.

2. Description of the Related Art

Skin is the largest organ that covers the surface of a human body and serves as the first guard line for protecting the human body from the invasion of foreign pathogens and external injuries. In a wound healing process, people have the ability of self-healing a small-area wound, but a wound dressing is generally required by patients having a large-area wound or a poor skin restoration ability such as diabetes patients or advanced cancer patients and provided for covering the wound to give a good environment for the healing and avoiding wound infections.

Clinically, a wound dressing such as a gauze or 3M™ Tegaderm™ artificial skin has a function simply limited to its covering onto the surface of the wound to prevent a secondary wound infection. Although such wound dressing provide a protection effect to a certain extent, yet it still cannot isolate the external environment and improve the leakage of wound tissue liquids that causes infections and inflammations, or fails to promote the restoration of new tissues of the wound. Therefore, it is an important research subject to find a way of preparing a wound dressing capable of protecting from infections and promoting the restoration of wound tissues to achieve the effect of healing the wound effectively.

At present, a relatively new wound dressing for healing wounds as disclosed in R.O.C. Pat. No. 1247614 is to use a polymer (such as chitosan and alginate copolymer) to combine with metal nanoparticles for enhancing the disinfection of the wound dressing and expediting the healing of the wound, or a wound dressing as disclosed in R.O.C. Pat. No. M365179 is to use a polyester compound to combine with nano silver and far infrared powder for effectively absorbing wound tissue liquids to achieve the effects of isolating external infections, improving the disinfection, and providing a good wound healing environment.

### SUMMARY OF THE INVENTION

In view of the shortcomings of the prior art, a primary objective of the present invention is to provide a wound dressing capable of overcoming the problems of the conventional wound dressing that cannot achieve the effects of avoiding wound infections and promoting wound tissue restoration.

To achieve the foregoing objective, the present invention provides a wound dressing, being a double-layer wound dressing, and comprising a polymer material layer and a porous carbon material layer. The polymer material layer is disposed at an upper layer of the double-layer wound dressing and comprises an anti-infective antibiotic or an anti-infective Chinese herb medicine for contacting with external air and blocking the invasion of foreign bacteria. The porous carbon material layer is disposed at a lower layer of the double-layer wound dressing and provided for attaching onto a wound surface.

Preferably, the porous carbon material layer can carry epithelial cells to expedite the wound healing.

Preferably, the porous carbon material layer further comprises an anti-adhesion polymer to reduce the production of adhesion condition.

To achieve the foregoing objective, the present invention also provides a wound dressing, comprising a polymer material layer having an anti-infective Chinese herb medicine for blocking the invasion of bacteria in the external environment.

In summation, the wound dressing of the present invention has one or more of the following advantages:

(1) The present invention is a functional wound dressing that combines an anti-infective antibiotic or a Chinese herb medicine with a porous carbon material to improve the disinfection and avoid the invasion of external pathogens, while improving the aggregation of skin-associated cells and stimulating the regeneration of Type I collagens so as to expedite the restoration of wounds and shorten the healing time.

(2) The wound dressing of the present invention adopts the porous carbon material with a good mechanical property for actively absorbing secreted tissue liquids, and the polymer material layer comprises an anti-infective antibiotic or a Chinese herb medicine that can destroy bacteria around the wound dressing. In addition, the porous carbon material can carry epithelial cells such as homogeneous dermal fibroblast, and a homogeneous dermal fibroblast transplantation can reduce autoimmune reactions to induce the aggregation of fibroblasts at the wound and provide the regeneration of cells concurrently, so as to expedite the wound healing.

(3) The wound dressing of the present invention is made of a material with good skin friendliness, good biocompatibility and a low price, and its preparation process requires no special instruments or equipments, and the produced wound dressing has good quality and reproducibility.

(4) The wound dressing of the present invention is a multifunctional wound dressing capable of achieving the anti-bacteria, inflammation resisting, cell collagen proliferating and wound healing effects.

### BRIEF DESCRIPTION OF THE DRAWINGS

FIGS. 1A to 1D are schematic views of a double-layer wound dressing in accordance with first to fourth embodiments of the present invention, respectively;

FIG. 2 shows SEM photos of RDF cells after being grown on activated carbon fibers for four days;

FIG. 3 is a graph of the area of a healed wound versus time of a double-layer wound dressing of the present invention and other different wound dressings;

FIG. 4 is a graph of the depth of a healed wound versus time of a double-layer wound dressing of the present invention and other different wound dressings;

FIG. 5 shows photos of H&E immunohistochemical stains of wound tissues on a double-layer wound dressing of the present invention and a gauze at different time;

FIG. 6 shows photos of Masson's Trichrome stains of wound tissues on a double-layer wound dressing of the present invention and a gauze at different times;

FIG. 7 is a histogram of contents of Type I collagen of wound tissues of a double-layer wound dressing of the present invention and a gauze at different times;

FIG. 8 is a graph of the area of a healed wound versus time of a wound dressing of the present invention and other different wound dressings; and

FIG. 9 is a graph of the depth of a healed wound versus time of a wound dressing of the present invention and other different wound dressings.

### DETAILED DESCRIPTION OF THE PREFERRED EMBODIMENTS

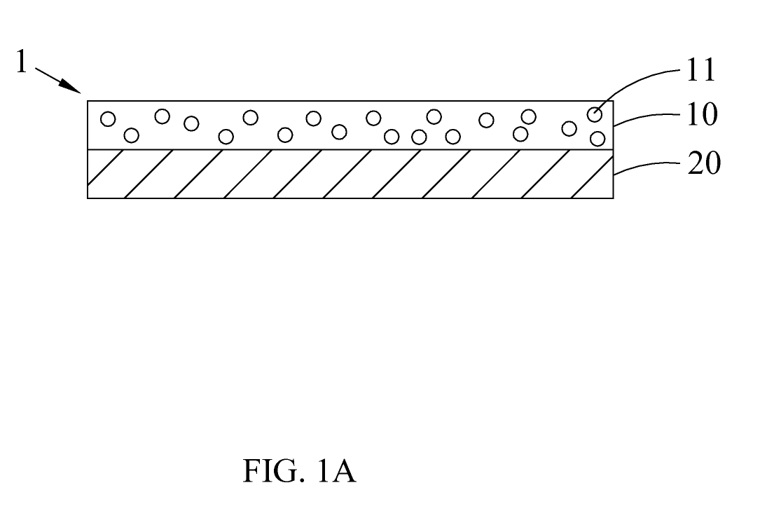

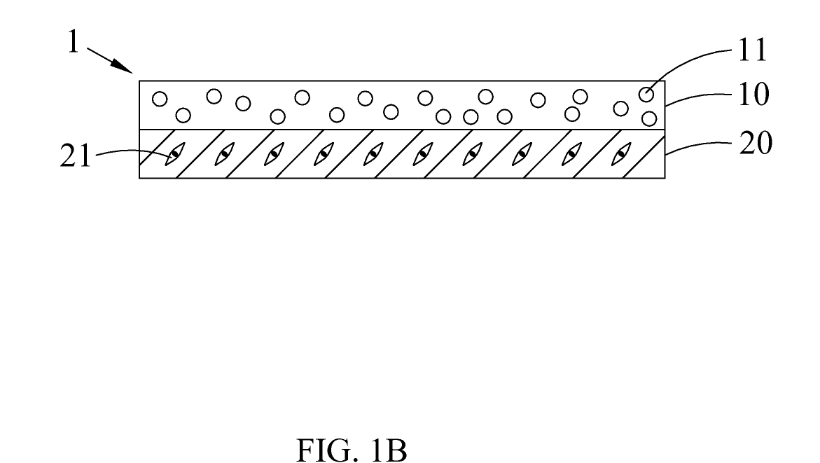

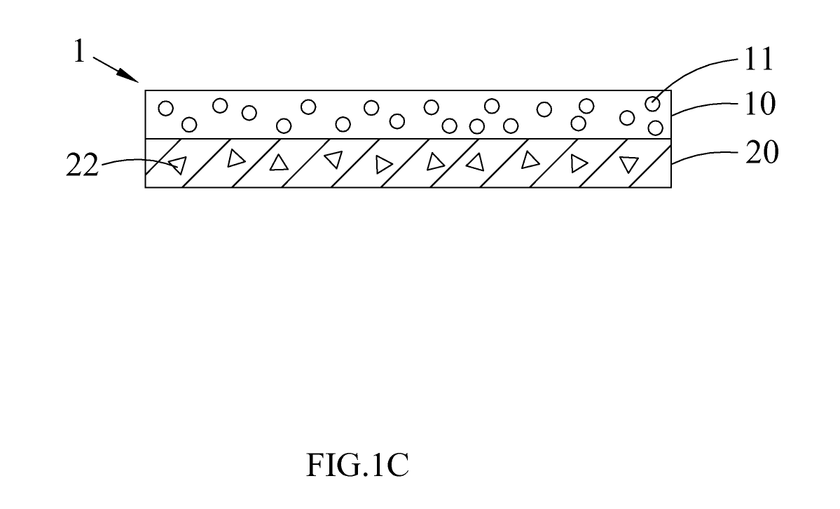

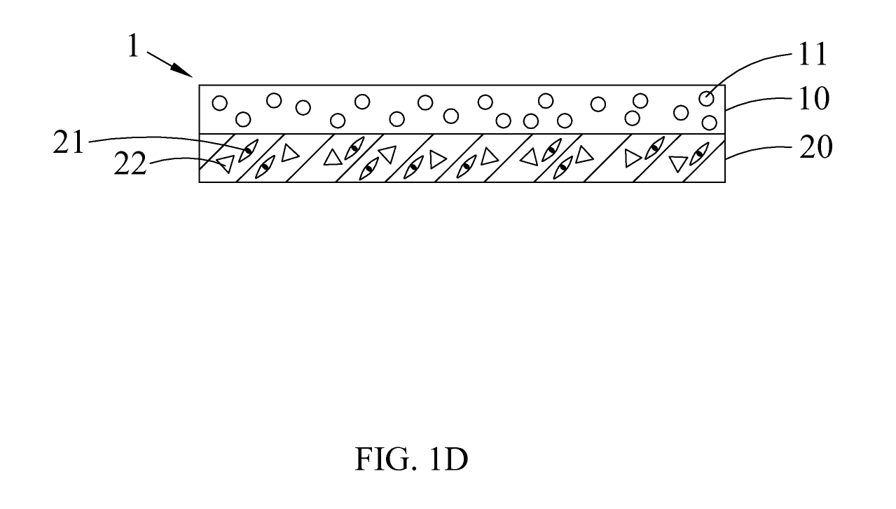

With reference to FIG. 1A for a schematic view of a wound dressing in accordance with a first embodiment of the present invention, the wound dressing is a double-layer wound dressing 1, comprising a polymer material layer 10 and a porous carbon material layer 20. The polymer material layer 10 is disposed at the upper layer of the double-layer wound dressing 1 and comprises an anti-infective antibiotic or Chinese herb medicine 11 for contacting external air and blocking the invasion of external bacteria. The porous carbon material layer 20 is disposed at the lower layer of the double-layer wound dressing 1 for attaching onto a wound surface and contacting with the wound. The wound dressing of the present invention can selectively have the design of a single-layer wound dressing (not shown in the figure) comprising a polymer material layer 10, and an anti-infective antibiotic or Chinese herb medicine 11 contained in the polymer material layer 10 for blocking the invasion of bacteria in the external environment and promoting the healing of the wound.

The polymer material layer 10 is made of gelatin, hyaluronic acid, collagen, polyglutamic acid, chitosan, fucoidan, sodium alginate, or a combination thereof, which has the advantages of a low price, good biocompatibility, high hydrophility and high moisture retainability. In addition, the polymer material layer 10 can be attached closely onto the porous carbon material layer 20, and has a very good anti-bacteria function to prevent infections from micro-organisms and very good moisture retainability and an appropriate penetration of water moisture while maintaining the moisture retainability of the wound dressing and the wound contact surface to prevent the adhesion of the wound dressing and the wound tissues so as to avoid secondary injury occurred during a dressing change, thus providing a good restoration environment for the wound.

The anti-infective antibiotic can comprise aminoglycosides such as gentamicin sulfate, macrolides such as erythromycin, chloramphenicols, tetracyclines, penicillins, mafenide acetate, or ceftazidime hydrate. Wherein, the antibiotic such as gentamicin can suppress the growth of bacteria effectively while stimulating the growth of Type I collagens of the wound tissues to achieve the effect of promoting the wound healing. The anti-infective Chinese herb medicine may be a shenjiyuhonggao paste extract, such as Danggui (i.e. Angelica sinensis), Zicao (i.e. Lithospermum erythrorhizon), Baizhi (i.e. Angelica dahurica), Gancao (i.e. Glycyrrhiza uralensis), and Xuejie (i.e. Bleeding stopper herb), or a combination thereof, capable of the effects of resisting inflammations and promoting the wound healing concurrently.

The porous carbon material layer may be made of a material in form of powder, fiber, or flake, and the material comprises activated carbon, graphite, carbon, or a combination thereof. For example, the material can be an activated carbon fiber, powdered bamboo-carbon, porous graphite (such as expanded graphite), or flakes formed by compressing the above.

When the double-layer wound dressing 1 is covered onto a wound, pores formed on the porous carbon material layer 20 are provided for adsorbing cells, enhancing the aggregations of cells, and stimulating the growth of collagens to expedite the restoration of the wound and shorten the healing time of the wound. The polymer material layer 10 located in the external layer of the double-layer wound dressing 1 can be provided for encapsulating the anti-infective antibiotic or Chinese herb medicine 11 and further preventing external micro-organisms and bacteria from infecting the wound.

With reference to FIG. 1B for a schematic view of a wound dressing in accordance with a second embodiment of the present invention, the wound dressing is a double-layer wound dressing 1 with the characteristics of the first embodiment of the present invention, and the porous carbon material layer 20 of the second embodiment can further carry skin-associated epithelial cells 21, such as keratinocytes, fibroblast cells, granulocytes, or a combination thereof. The epithelial cells 21 can be loaded in the porous carbon material layer 20 by any appropriate method such as a steeping, soaking, immersing or dropping method, and the epithelial cells 21 can be carried in the porous carbon material layer 20 in any appropriate form. The porous carbon material layer 20 is made of a material having good skin friendliness, such that when the porous carbon material layer 20 is covered onto the wound, the epithelial cells 21 adsorbed into the pores of the porous carbon material layer 20 can suck the cells of the wound tissues to form a cell aggregation and also can promote the growth of the cell collagens to expedite the wound healing when the wound dressing is applied.

With reference to FIG. 1C for a wound dressing in accordance with a third embodiment of the present invention, the wound dressing is a double-layer wound dressing 1 with the characteristics of first embodiment, the porous carbon material layer 20 of the third embodiment further comprises an anti-adhesion polymer 22 including but not limited to gelatin, hyaluronic acid, collagen, polyglutamic acid, chitosan, fucoidan, sodium alginate, or a combination thereof to reduce the adhesion condition occurred during the process of restoring the skin tissues. The concentration of hyaluronic acid is preferably 5 g/ml to 50 g/ml. The concentration of gelatin is preferably 25 g/ml to 200 g/ml. The concentration of polyglutamic acid is preferably 5 g/ml to 50 g/ml.

With reference to FIG. 1D for a wound dressing in accordance with a fourth embodiment of the present invention, the wound dressing of the fourth embodiment is a double-layer wound dressing 1 with the characteristics of the second embodiment, and the double-layer wound dressing 1 of the fourth embodiment also attaches an anti-adhesion polymer 22 comprising but not limited to gelatin, hyaluronic acid, collagen, polyglutamic acid, chitosan, fucoidan, sodium alginate, or a combination thereof onto the porous carbon material before the epithelial cells 21 are added, so as to reduce the occurrence of adhesion during the process of restoring the skin tissues.

The preparation of a polymer material layer of a wound dressing in accordance with a first embodiment the present invention and the anti-bacteria capability test are elaborated as follows. In this embodiment, the polymer material layer is prepared by dissolving Type A gelatin (blood number 300) and polyglutamic acid (y-PGA) in a PBS buffer, and the final concentrations of the two are 10% (w/v) and 3.75% (w/v) respectively, and then a gentamicin antibiotic with a concentration of 0.05% is added, and a crosslinking agent (genipin) with a concentration of 0.05% (w/v) is mixed uniformly, and 10 ml of the uniformly mixed solution is poured into a plastic Petri dish with a diameter of 9 cm, and then placed into an oven at 37° for a bake-drying process after being cross-linked at room temperature for 10 hours, and finally disinfected by an ultraviolet lamp overnight.

In the anti-bacteria capability test, three kinds of common bacteria occurred in clinical infection are selected, and these three bacteria are Escherichia coli, Pseudomonas aeruginosa and Staphylococcus aureus. The foregoing produced polymer material layer is cut into a plurality of test strips, each having a size with a diameter of approximately 10 mm, and covered onto agar plates for cultivating the aforementioned three kinds of bacteria respectively, and placed and cultivated in an environment at 37° for 24 hours for the observation of the growth of bacteria around the polymer material layer test strip. Experiment results show that the polymer material layer wrapped with gentamicin has a good bacteria suppression effect on a solid bacteriostatic ring cultivation test of three kinds of bacteria strains, and the sizes of the bacteriostatic rings are given as follows: 2.05±0.054 cm for Escherichia coli, 3.06±0.114 cm for Pseudomonas aeruginosa and 1.93±0.057 cm for Staphylococcus aureus.

In this embodiment, the double-layer wound dressing of the present invention is prepared by dissolving Type A gelatin (blood number 300) and polyglutamic acid (y-PGA) into a PBS buffer, and the final concentrations of the two are 10% (w/v) and 3.75% (w/v) respectively, and then a gentamicin antibiotic with a concentration of 0.05% is added, and a crosslinking agent (genipin) with a concentration of 0.05% (w/v) is mixed uniformly, and 10 ml of the uniformly mixed solution is poured into a plastic Petri dish with a diameter of 9 cm, and then placed into an oven at 37° for a bake-drying process after being cross-linked at room temperature for 10 hours to produce a polymer material layer. The activated carbon fiber for forming the porous carbon material layer is covered onto the polymer material layer. During the covering process, it is necessary to prevent the gelatin/polyglutamic acid solution from permeating from the undried polymer material layer and thus clocking the pore of each the activated carbon fiber. Finally, the double-layer wound dressing is placed into an oven at 37° for a bake-drying process and disinfected by an ultraviolet lamp to produce the double-layer wound dressing of the present invention.

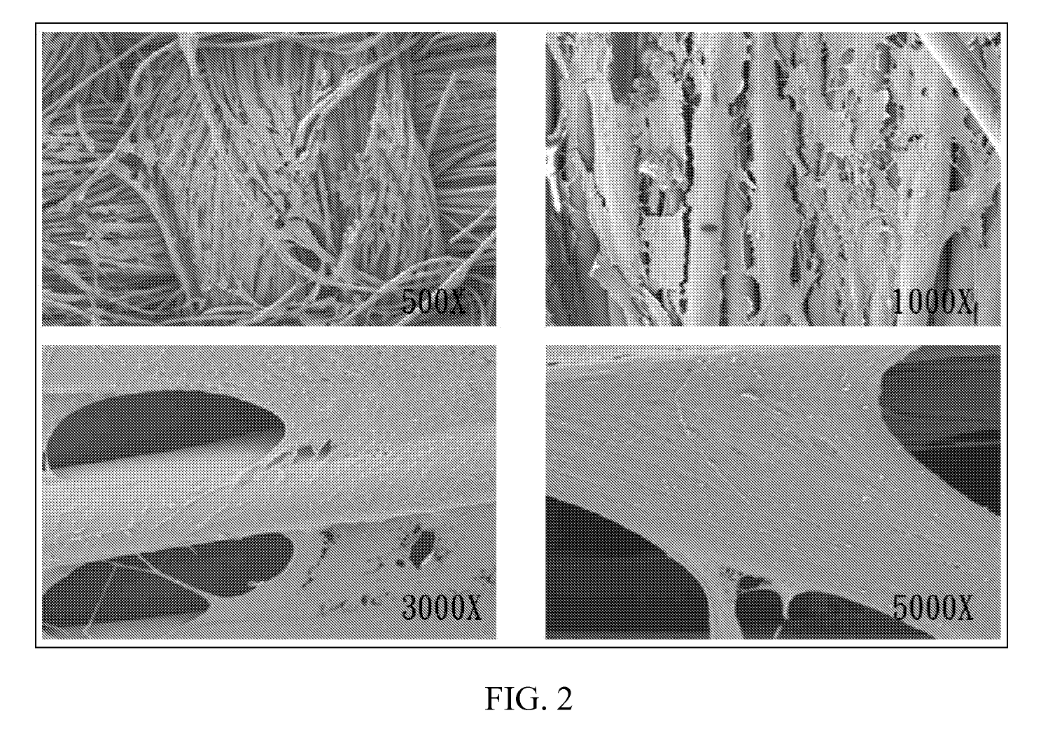

The double-layer wound dressing with RDF cells in accordance with the present invention is prepared by the aforementioned method, and then cut into a plurality of test strips with a diameter of 20 mm, and the test strip is laid flatly at the bottom of a cell culture medium, and then 5×10⁵ epithelial cells (the cells used in this embodiment are rat dermal fibroblast cells (RDF cells) are implanted into the porous carbon material layer of the test strip and cultivated in a cell culture incubator for four days to prepare the double-layer wound dressing with the RDF cells. With reference to FIG. 2 for SEM photos of RDF cells after being grown on activated carbon fibers for four days, the RDF cells are attached closely onto the surface of the activated carbon fiber and can be grown to the position of single fibers and bridges between fibers after the RDF cells are grown on the porous carbon material layer made of the activated carbon fiber for four days.

The effect of the double-layer wound dressing with RDF cells in accordance with the present invention on the wound healing is described as follows. In this test, four different wound dressings are adopted, and their conditions and pre-processing methods are given below: (1) a gauze available in the market: The gauze is cut into 2×2 cm² and disinfected by an ultraviolet lamp overnight before an animal operation takes place; (2) a double-layer wound dressing without RDF cells: The foregoing produced double-layer wound dressing is cut into 2×2 cm² and disinfected by an ultraviolet lamp before an animal experiment takes place, and then dipped into a PBS buffer; (3) a double-layer wound dressing with RDF cells: The foregoing produced double-layer wound dressing is cut into 2×2 cm² and disinfected by an ultraviolet lamp, and the wound dressing is moved to a culture dish, and 5.5×10⁵ cells are cultivated for four days before an animal experiment takes place, and a PBS buffer is used for rinsing the surface of the wound dressing; and (4) a 3M wound dressing available in the market: this wound dressing is cut into 2×2 cm² and then the animal experiment is performed.

This test adopts Sprague-Dawley rats for the animal experiment. In the operation, a general anesthesia is performed to a rat, and then a wound of approximately 1×1 cm² is cut separately on both sides of the rat's back, and wound dressings of different conditions are sewed onto the wounds at the rat's back. After the operation ends, an elastic bandage is used to wrap the wounds and a net bandage is used for fixing the elastic bandage, and observations are taken after different numbers of days (such as 2, 4, 8 and 12 days), and a camera with a fixed focal length at a fixed distance is used for taking the photos of the rat's wound conditions in the observation process, and Image-Pro® Plot 6.0 software is used for measuring the area of the wound and the wound healing depth, and five positions of a same wound are taken for the measurement and an average is computed. The wound healing percentage for the original area or depth of the wound is 100%, and the wound restoration is estimated for analyzing the wound healing after the operation takes place, and the following formulas are used for the analysis:

Wound Closure % (Area)=[(Area_{day0}−Area_{dayn})/Area_{day0}]×100; and

Wound Closure % (Depth), [(Depth_{day0}−Depthd_{ayn})/Depth_{day0}]×100.

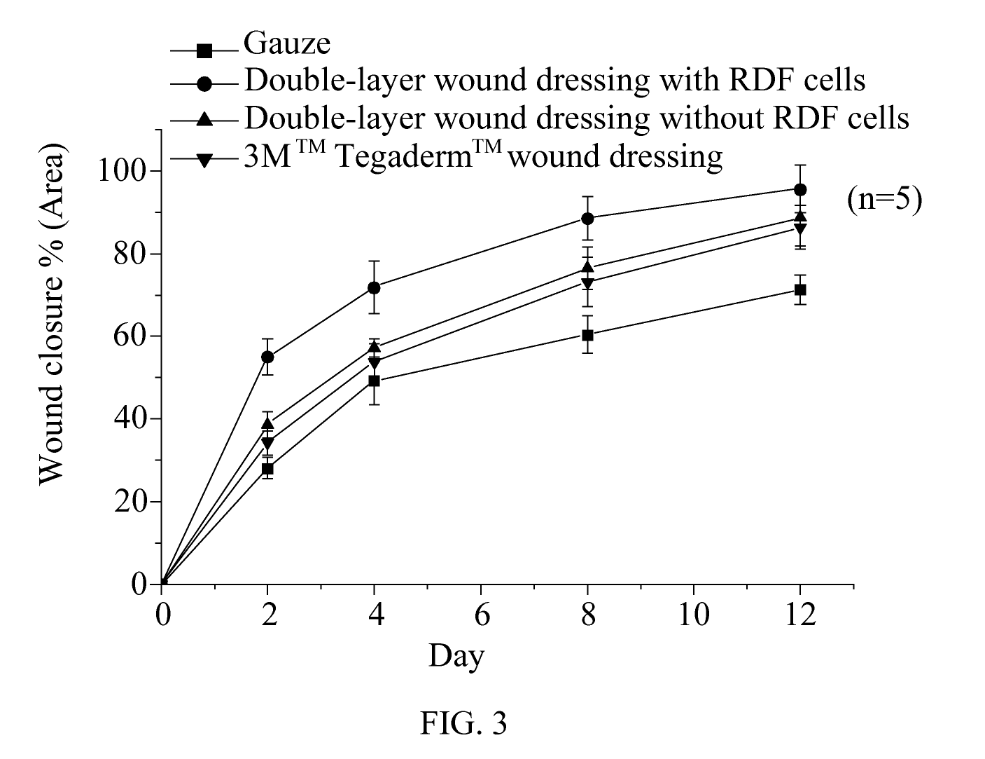

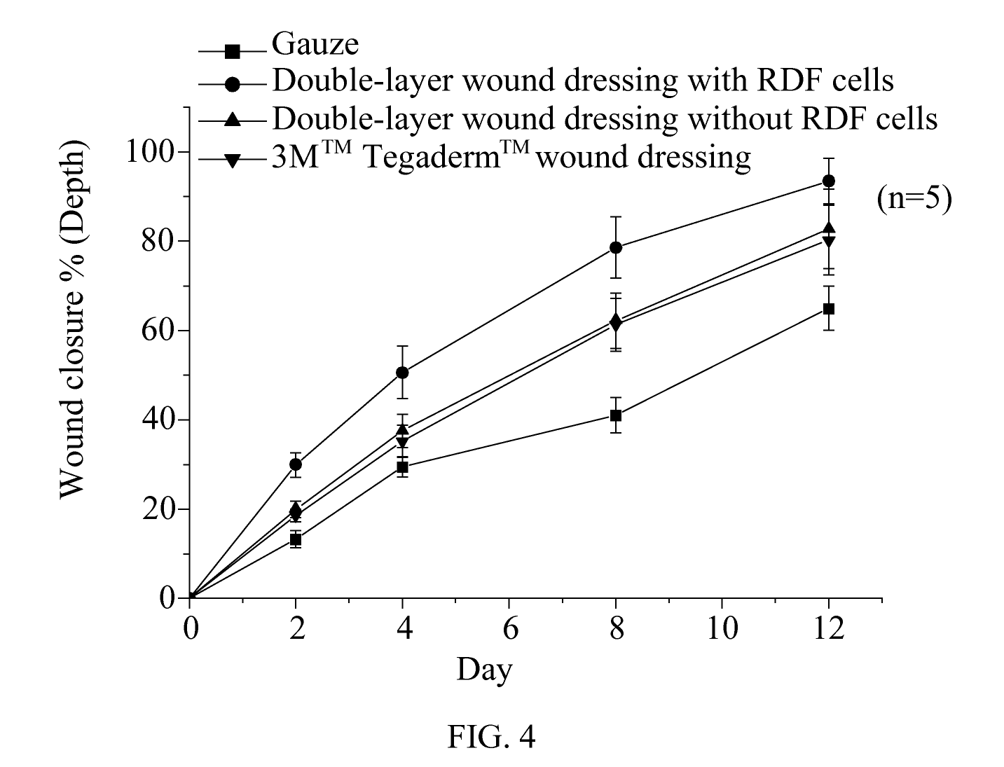

With reference to FIG. 3 for a graph of the area of a healed wound versus time of a double-layer wound dressing of the present invention and other different wound dressings, the wound restoration capability of the double-layer wound dressing with RDF cells reaches 55±4.46% on the 2^{nd} day of the wound restoration process, and such wound healing capability is much better than those of the 3M™ Tegaderm™ (34.3±2.98%) and gauze (28.1±2.58%) wound dressings. In an observation taken on the 12^{th} day after the operation takes place, the double-layer wound dressing with RDF cells has a wound restoration area of 94.8±7.99%, showing a much better wound healing capability than other wound dressings. The wound restoration also shows the same result as to the wound healing depth as shown in FIG. 4, and the wound healing depth of the double-layer wound dressing with RDF cells is 29.9±2.74% on the 2^{nd} day, and the 3M™ Tegaderm™ wound dressing available in the market achieves a wound healing depth of 18.5±1.42%, and the gauze achieves a wound healing depth of 13.3±1.91%, and the aforementioned wound healing depths have a significant difference.

On the 12^{th} days after the operation takes place, the wound healing depth of the double-layer wound dressing with RDF cells reaches up to 93.5±5.1%. In summation, the results show that both of the double-layer wound dressing with RDF cells of the present invention and other wound dressings play an important role for the tissue restoration and wound restoration in the wound restoration process, but the wound dressing of the present invention has the effect of expediting the wound healing.

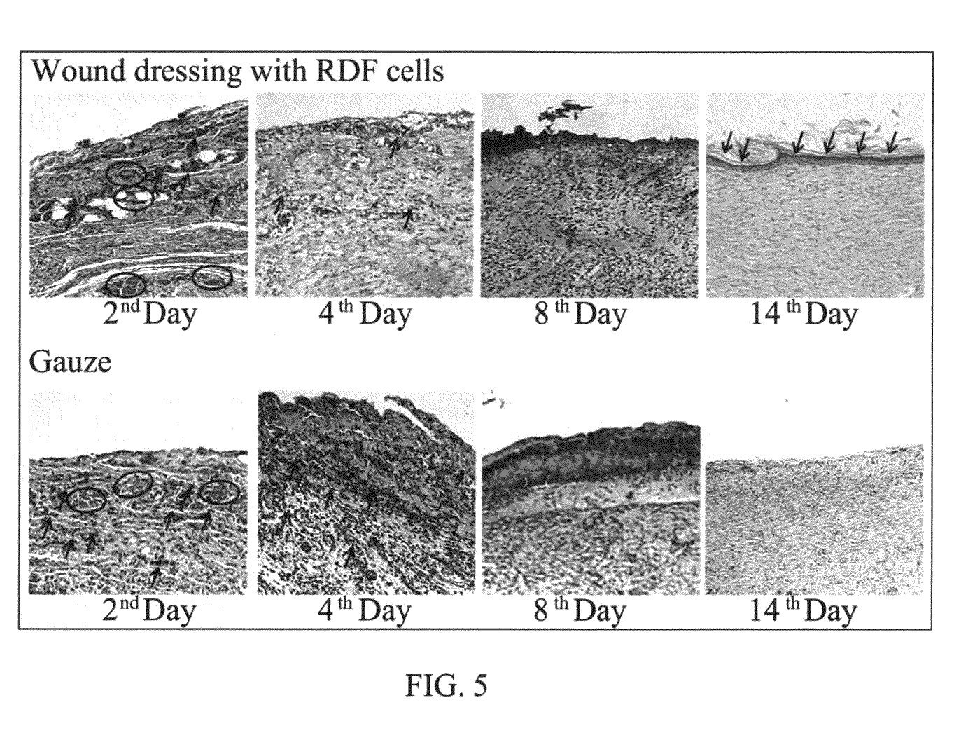

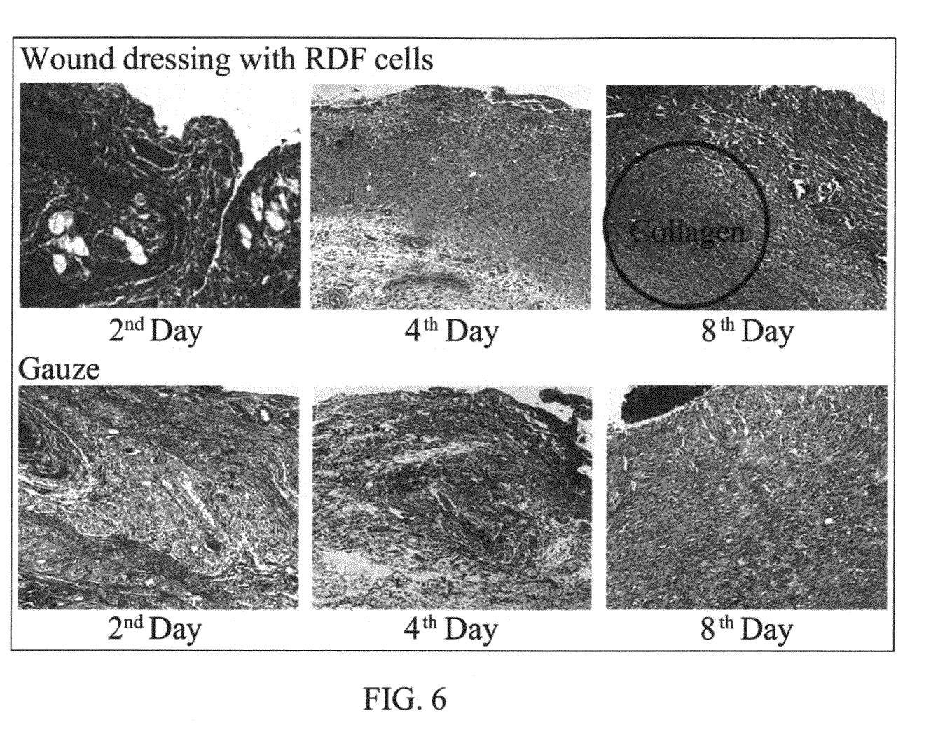

In an observation of the tissues after the wound is healed, the immunohistochemical staining (H&E staining) of the wound healing is applied for the observations on the 2, 4, 8 and 14 days to see the restoration of the inflamed cells and the growth of new blood vessels, and the results (with an amplification of 100×) are shown in FIG. 5. From the observation of the immunohistochemical staining, the wound restoration is still in the blood clotting and inflammation period on the 2^{nd} day after the operation, and the H&E tissue staining show the performance of the inflamed cells (purple granules indicated by the arrowhead) and the growth of the new blood vessel (red dotted area indicated by the circle). As time passes, the double-layer wound dressing with RDF cells restores the wound, and the number of inflamed cells decreases, and collagens (indicted in the pink red area) are grown gradually. On the 14^{th} day after the wound restoration, a thin layer of stratum corneum is formed in the skin tissues. On the other hand, the control group (gauze) still shows an inflammation conditions of the wound tissue on the 8^{th} day after the wound restoration, and no stratum corneum can be observed (at the position indicated by the arrowhead) yet on the 14^{th} day. In the Masson trichrome staining results (observed with an amplification of 100×) of the wound tissues as shown in FIG. 6 show that the double-layer wound dressing with RDF cells gives an obvious new growth of collagens on the 2^{nd}, 4^{th} and 8^{th} days after the operation, but the gauze available in the market does not have any significant finding.

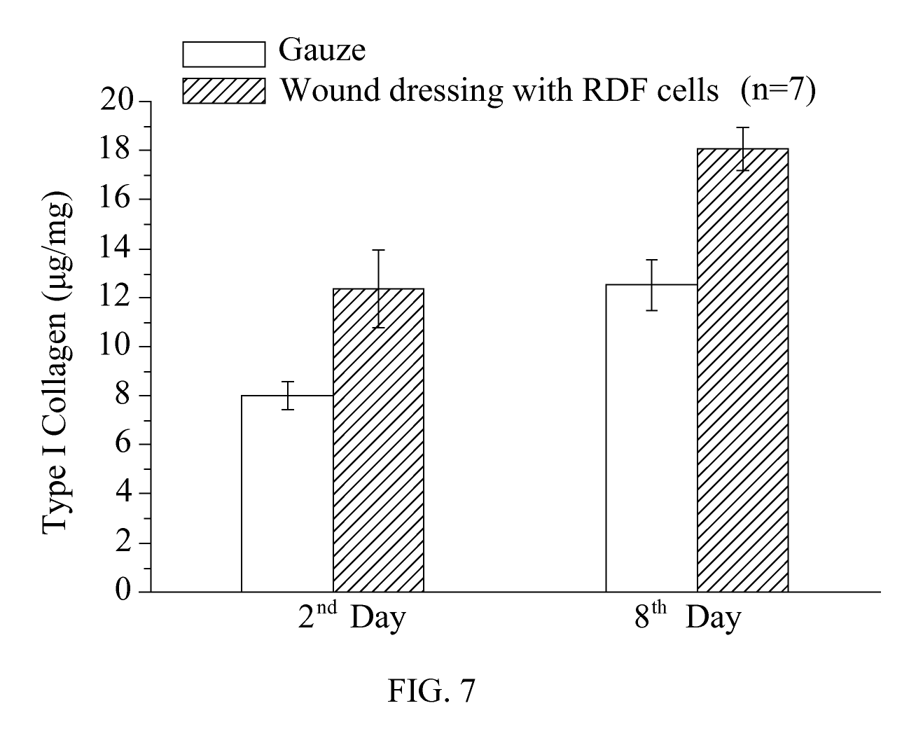

Type I collagen shows up in the wound healing process. This experiment uses a rat's type I collagen detection kit to measure the difference of forming quantities of Type I collagens in the skin tissues. In FIG. 7, the double-layer wound dressing with RDF cells and the gauze are compared on the 2^{nd} day, the quantities of formed Type I collagens are 12.35±1.57 μg/mg and 8±0.59 μg/mg respectively, and it shows a significant difference between these two numbers. On the 8^{th} day, the quantity of formed Type I collagen by the double-layer wound dressing reaches 18.07±0.88 μg/mg, which is much greater than the control group (12.5±1.03 ng/mg). Therefore, the quantitative results show that the double-layer wound dressing with RDF cells obviously forms Type I collagens to assist healing the wound in the wound healing process.

In summation of the aforementioned results of the cell and animal experiments, the double-layer wound dressing having an upper layer which is a polymer material layer combined with an antibiotic (gentamicin) and a lower layer which is a porous carbon material layer provided for with RDF cells has a significant healing effect for the restoration of the wound tissues and the repair of the wound, and thus this wound dressing has a high potential development on clinical application, compared with the general gauzes and 3M™ Tegaderm™ wound dressing available in the market.

The preparation of a wound dressing with a Chinese herb medicine in accordance with a second embodiment the present invention is described as follows:

In the preparation of the wound dressing with the Chinese herb medicine in accordance with this embodiment of the present invention, the wound dressing is a polymer material layer produced by dissolving Type A gelatin (blood number 300) and polyglutamic acid (y-PGA) into a PBS buffer, and the final concentrations of the two are 10% (w/v) and 3.75% (w/v) respectively, and then a shenjiyuhonggao paste extract is added, wherein Danggui (i.e. Angelica sinensis), Xuejie (being a kind of bleeding stopper herb), Zicao (i.e. Lithospermum erythrorhizon), Gancao (i.e. Glycyrrhiza uralensis), and Baizhi (i.e. Angelica dahurica) preferably have a concentration of 0.5 g/ml to 5 g/ml respectively, and a crosslinking agent (genipin) with a concentration of 0.05% (w/v) is mixed uniformly, and 10 ml of the uniformly mixed solution is poured into a plastic Petri dish with a diameter of 9 cm, and then placed into an oven at 37° C. for a bake-drying process after being cross-linked at room temperature for 10 hours, and finally disinfected by an ultraviolet lamp overnight.

The effect of the wound dressing with Chinese herb medicine in accordance with the present invention on the wound healing is described as follows. In this test, three different types of wound dressings are adopted, and their conditions and pre-processing methods are given below: (1) a gauze available in the market: The gauze is cut into 2×2 cm² and disinfected by an ultraviolet lamp overnight before an animal operation takes place; (2) a wound dressing with a Chinese herb medicine: The foregoing produced wound dressing is cut into 2×2 cm² and disinfected by an ultraviolet lamp, and the wound dressing is dipped into a PBS buffer before the animal experiment takes place; and (3) a 3M™ Tegaderm™ wound dressing available in the market: this wound dressing is cut into 2×2 cm² and then the animal experiment is performed.

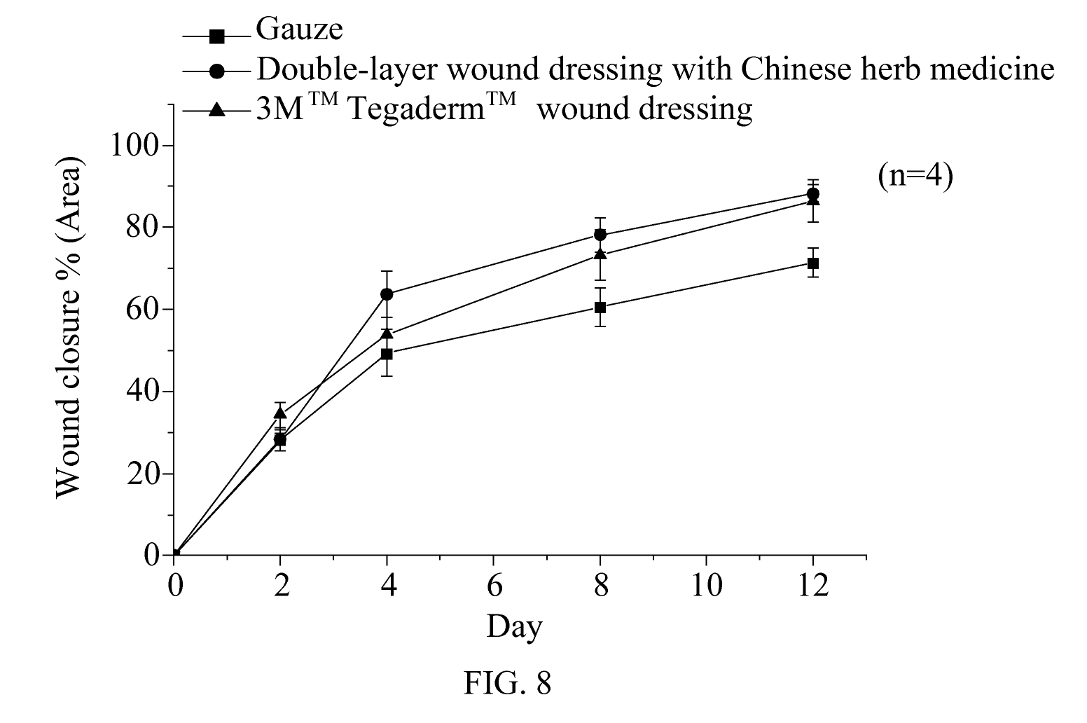

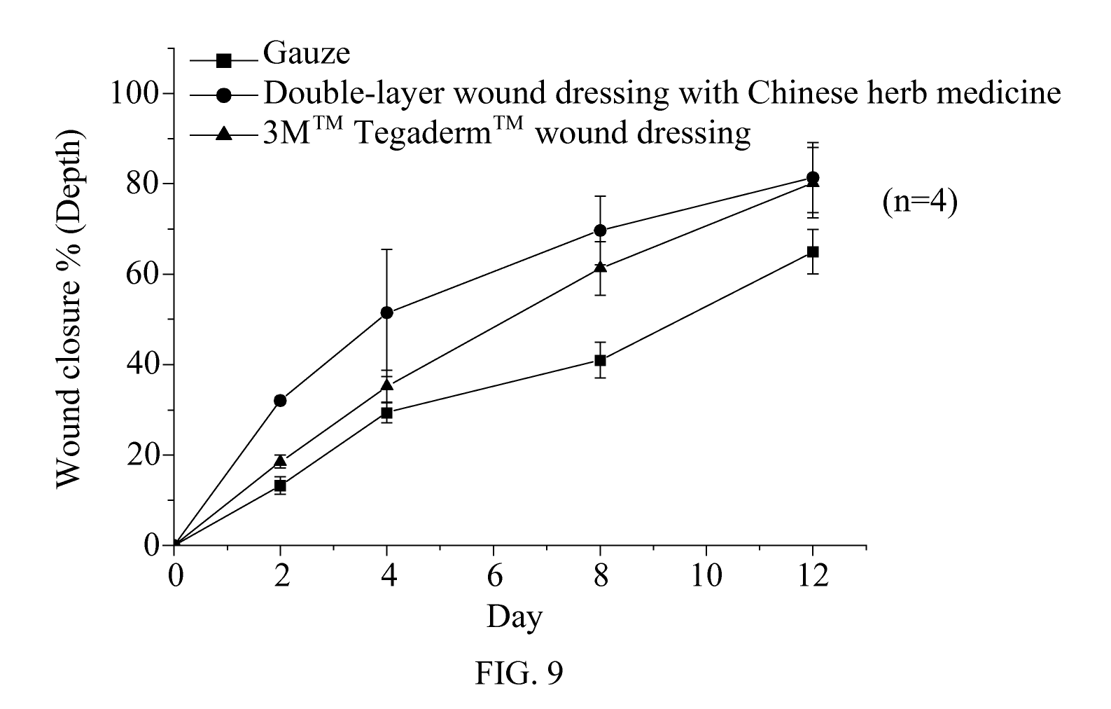

This test adopts Sprague-Dawley rats for the animal experiment. In the operation, a general anesthesia is performed to a rat, and then a wound of approximately 1×1 cm² is cut separately on both sides of the rat's back, and wound dressings of different conditions are sewed onto the wounds at the rat's back. After the operation ends, an elastic bandage is used to wrap the wounds and a net bandage is used for fixing the elastic bandage, and observations are taken after different numbers of days (such as 2, 4, 8 and 12 days), and a camera with a fixed focal length at a fixed distance is used for taking the photos of the rat's wound conditions in the observation process, and Image-Pro® Plot 6.0 software is used for measuring the area of the wound and the wound healing depth, and five positions of a same wound are taken for the measurement and an average is computed. The wound healing percentage for the original area or depth of the wound is 100%, and the wound restoration is estimated for analyzing the wound healing after the operation takes place. The formulas for the evaluation are the same as above, and thus will not be repeated here. The results of the experiments as shown in FIGS. 8 and 9 indicate that the wound dressing with the Chinese herb medicine obviously has a better wound healing capability than other wound dressings. As to the wound restoration, the same result applies to the wound healing depth. In summation of the aforementioned results, both of the wound dressing with the Chinese herb medicine of the present invention and other wound dressings play an important role for the tissue restoration and wound restoration in the wound restoration process, but the wound dressing of the present invention has the effect of expediting the wound healing.

What is claimed is:

1. A wound dressing, being a double-layer wound dressing, and comprising:
a polymer material layer, comprising an anti-infective antibiotic or an anti-infective Chinese herb medicine, disposed at an upper layer of the double-layer wound dressing, and used for contacting external air and blocking an invasion of foreign bacteria; and
a porous carbon material layer, disposed at a lower layer of the double-layer wound dressing, and used for attaching onto a wound surface.

2. The wound dressing of claim 1, wherein the polymer material layer is made of a material selected from a group consisting of gelatin, hyaluronic acid, collagen, polyglutamic acid, chitosan, fucoidan, sodium alginate, and a combination thereof.

3. The wound dressing of claim 1, wherein the anti-infective antibiotic comprises aminoglycosides, macrolides, chloramphenicols, tetracyclines, or penicillins.

4. The wound dressing of claim 1, wherein the anti-infective antibiotic comprises gentamicin sulfate, mafenide acetate, or ceftazidime hydrate.

5. The wound dressing of claim 1, wherein the anti-infective Chinese herb medicine comprises a shenjiyuhonggao paste extract.

6. The wound dressing of claim 5, wherein the shenjiyuhonggao paste extract comprises Danggui, Zicao, Baizhi, Gancao, Xuejie, or a combination thereof.

7. The wound dressing of claim 1, wherein the porous carbon material layer is made of a material in form of a powder, a fiber or a flake.

8. The wound dressing of claim 7, wherein the porous carbon material layer is made of a material selected from a group consisting of activated carbon, graphite, carbon, and a combination thereof.

9. The wound dressing of claim 1, wherein the porous carbon material layer carries epithelial cells.

10. The wound dressing of claim 9, wherein the epithelial cells are skin-associated and comprise keratinocytes, fibroblasts, granulocytes, or a combination thereof.

11. The wound dressing of claim 1, wherein the porous carbon material layer comprises an anti-adhesion polymer.

12. The wound dressing of claim 11, wherein the anti-adhesion polymer comprises gelatin, hyaluronic acid, collagen, polyglutamic acid, chitosan, fucoidan, sodium alginate, or a combination thereof.

13. A wound dressing, comprising:
a polymer material layer, comprising an anti-infective Chinese herb medicine, for blocking an invasion of foreign bacteria.

14. The wound dressing of claim 13, wherein the anti-infective Chinese herb medicine is a shenjiyuhonggao paste extract.

15. The wound dressing of claim 14, wherein the shenjiyuhonggao paste extract comprises Danggui, Zicao, Baizhi, Gancao, Xuejie, or a combination thereof.
